(19) United States
(12) Patent Application Publication
(10) Pub. No.: US 2026/0060768 A1
(43) Pub. Date: Mar. 5, 2026

(54) MEDICAL ROBOT SYSTEM, METHOD OF CONTROLLING, AND COMPUTER-READABLE MEDIUM

(71) Applicant: ASAHI SURGICAL ROBOTICS CO., LTD, Kashiwa-shi (JP)

(72) Inventors: Takehiro ANDO, Chiba (JP); Keita AWANO, Chiba (JP)

(73) Assignee: ASAHI SURGICAL ROBOTICS CO., LTD, Kashiwa-shi (JP)

(21) Appl. No.: 19/386,806

(22) Filed: Nov. 12, 2025

Related U.S. Application Data
(63) Continuation of application No. PCT/JP2023/017808, filed on May 11, 2023.

(51) Int. Cl. A61B 34/37; A61B 34/30

(52) U.S. Cl. CPC A61B 34/37; A61B 2034/301; A61B 2034/306

(57) ABSTRACT

A medical robot system includes a plurality of robot arms, a surgeon operation portion, and a controller. When an instruction to operate one of the robot arms and a medical instrument attached thereto is received, for each of the robot arms and each of medical instruments attached thereto, the controller is configured to set an inclusion area encompassing the robot arm and a portion on a side closer to the robot arm with respect to a shaft portion, and does not set an inclusion area encompassing the shaft portion and a portion on a distal end side with respect to the shaft portion, and the controller is configured to determine whether the inclusion area after moving the inclusion area corresponding to the one of the robot arms and the medical instrument attached thereto interferes with the inclusion areas corresponding to the remaining robot arms and the medical instruments attached thereto.

### CROSS REFERENCE TO RELATED APPLICATIONS

This application is a continuation application of International Application No. PCT/JP2023/017808, filed May 11, 2023. The disclosure of the prior application is incorporated herein by reference in the entirety.

### TECHNICAL FIELD

The present disclosure relates to a medical robot system, a method of controlling, and a computer-readable medium.

### BACKGROUND

Japanese Unexamined Patent Publication No. 2000-300579 discloses a multifunctional manipulator configured such that even when multiple manipulators including an endoscope, a treatment instrument, and the like are simultaneously operated within a body cavity, the manipulators do not interfere with one another.

### SUMMARY

In the above-mentioned multifunctional manipulator, it is not necessarily required to avoid interference (collision) between the endoscope and the treatment instrument located within the body cavity, and there is a situation where attempting to avoid interference may reduce the degree of freedom of movement of each of the endoscope and the treatment instrument.

The present disclosure is to provide a medical robot system, a method of controlling, and a computer-readable medium which are capable of preventing interference of a robot arm while ensuring freedom of movement of a medical instrument located in a body cavity.

A medical robot system according to an aspect of the present disclosure includes a plurality of robot arms, each having a medical instrument attached thereto; a surgeon operation portion that is configured to be directly operated by a surgeon so as to operate the robot arms and the medical instruments; and a controller that is configured to control the robot arms and the medical instruments based on an operation of the surgeon operation portion operated by the surgeon. Each of the medical instruments have a shaft portion inserted into a body cavity. When an instruction to operate one of the robot arms and the medical instrument attached thereto is received, for each of the robot arms and each of the medical instruments attached thereto, the controller is configured to set an inclusion area encompassing the robot arm and a portion on a side closer to the robot arm with respect to the shaft portion, and does not set an inclusion area encompassing the shaft portion and a portion on a distal end side with respect to the shaft portion, and the controller is configured to determine whether or not the inclusion area after moving the inclusion area corresponding to the one of the robot arms and the medical instrument attached thereto interferes with the inclusion areas corresponding to the remaining robot arms and the medical instruments attached thereto.

### BRIEF DESCRIPTION OF DRAWINGS

FIG. 1 is a diagram illustrating a functional configuration example of a medical robot system according to an embodiment.

FIG. 2 illustrates a robot arm portion, a patient, a surgeon, and an operating table when using the medical robot system of FIG. 1.

FIG. 3 shows a flow chart relating to an operation of a robotic medical instrument using a handheld medical instrument executed by a controller of FIG. 1.

FIG. 4 is an explanatory view of setting of inclusion areas for a horizontal robot arm, a linear motion robot arm, and the robotic medical instrument of FIG. 2.

### DETAILED DESCRIPTION

A medical robot system and a method of controlling, and a program according to embodiments of the present disclosure will be described with reference to the drawings below. However, the disclosed embodiments are not limited to the embodiments illustrated in the drawings.

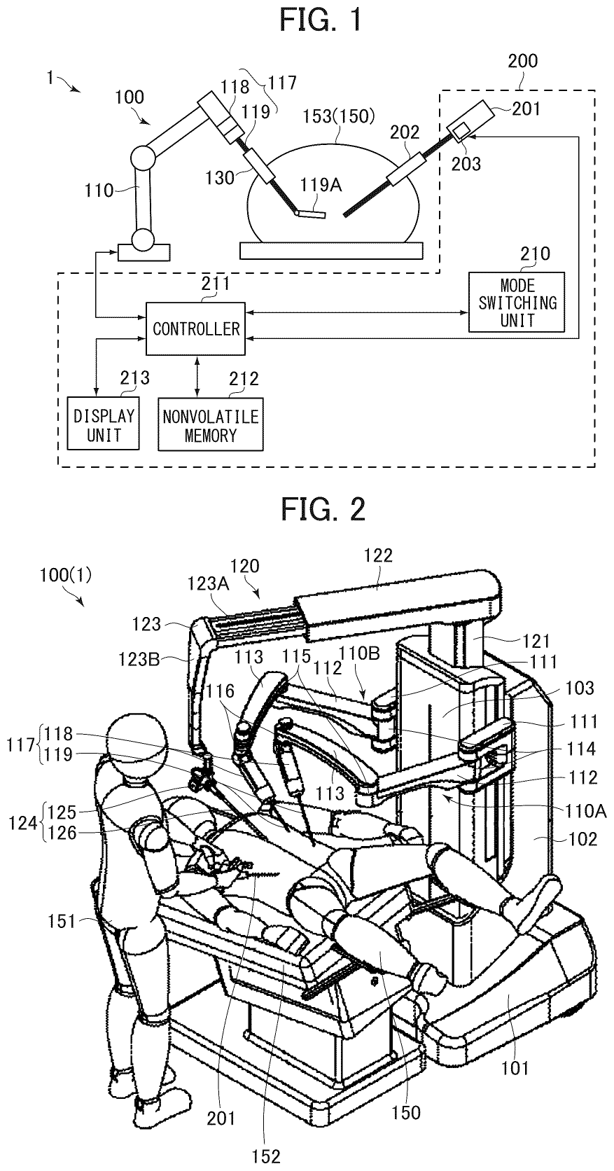

FIG. 1 is a diagram illustrating a functional configuration example of a medical robot system 1 according to an embodiment. Note that one or more functional blocks illustrated in FIG. 1 may be implemented by hardware such as an ASIC or a programmable logic array (PLA), or may be implemented by a programmable processor such as a CPU or an MPU executing software. In addition, the one or more functional blocks may be implemented by a combination of software and hardware. Accordingly, in the following description, even when different functional blocks are described as operating entities, the same hardware may serve as an entity that implements the functional blocks.

The medical robot system 1 includes a robot arm portion 100 and a surgery support device 200. The robot arm portion 100 controls a posture of a surgical instrument and an end effector inserted into a body cavity of a patient through an outer tube 130. The surgery support device 200 measures an insertion angle and an insertion depth of a surgical instrument (hereinafter, also referred to as a handheld medical instrument) that is inserted into the body cavity by a surgeon and actually used for a surgical operation. The surgery support device 200 controls the robot arm portion 100 for controlling the posture of the surgical instrument and the end effector (hereinafter, also collectively referred to as a robotic medical instrument) according to the measurement results. The surgeon can operate a handheld medical instrument 201 to, for example, alternately switch between: a procedure of, for example, incising a part of an organ with an electrosurgical knife; and control of the postures of a robotic medical instrument 117 and an end effector 119A (for example, traction of an organ with forceps). Consequently, the surgeon can perform, for example, an electrosurgical procedure and traction of an organ with forceps alone.

FIG. 2 illustrates the robot arm portion 100, a patient 150, a surgeon 151, and an operating table 152 when using the medical robot system 1. The robot arm portion 100 includes a plurality of frames (bases) 101 to 103, two horizontal robot arms 110A and 110B, and one linear motion robot arm 120.

The first frame 101 includes an active wheel or a passive wheel, or both, so that the patient 150 and the robot arm portion 100 can be arranged at an appropriate distance. The second frame 102 is a frame fixed above the first frame 101. The third frame 103 is connected so as to have a degree of freedom only in a direction perpendicular to the second frame 102. Two three-axis horizontal articulated robot arms (simply referred to as the horizontal robot arms 110A and 110B) having joints each having a degree of freedom in the horizontal direction are attached to the third frame 103. In addition, a three-axis linear motion articulated robot arm (also simply referred to as the linear motion robot arm 120) is connected to the distal end of the third frame 103 by a member having a degree of freedom only in the vertical direction.

Each of the horizontal robot arms 110A and 110B includes a first link 111, a second link 112, a third link 113, a first joint 114, a second joint 115, and a third joint 116. The robotic medical instrument 117 is attached to the distal end of each of the horizontal robot arms 110A and 110B. The first link 111 and the second link 112 are connected by the first joint 114 which is an active joint having a degree of freedom only in the horizontal rotation. The second link 112 and the third link 113 are similarly connected by the second joint 115 which is an active joint having a degree of freedom only in horizontal rotation. The robotic medical instrument 117 is connected to the distal end of the third link 113 via the third joint 116 having a gimbal mechanism configured to be rotatable about two axes. The first link 111 has an active joint having a degree of freedom in the vertical direction (longitudinal direction of the third frame 103). Therefore, the second link 112 and the third link 113 are movable in the vertical direction by the first link 111. Consequently, the robotic medical instrument 117 is allowed to move three-dimensionally, and an insertion angle and an insertion depth of the robotic medical instrument 117 into the body cavity are thus controlled.

The robotic medical instrument 117 includes an actuator unit 118 and a surgical instrument shaft 119. The actuator unit 118 is connected to the third joint 116 and has a plurality of motors each generating an independent rotational drive. The actuator unit 118 has a substantially hollow cylindrical shape and includes a drive adapter that transmits drive power to the end effector 119A (see FIG. 1). The surgical instrument shaft 119 is detachably connected to the distal end of the actuator unit 118. The end effector 119A is provided at the distal end of the surgical instrument shaft 119. The position and posture of the end effector 119A are controlled by drive power from the actuator unit 118. When performing a surgical operation, a part of the surgical instrument shaft 119 of the robotic medical instrument 117 and the end effector 119A are inserted into the body cavity of the patient 150 lying on the operating table 152. The robotic medical instrument 117 includes, for example, forceps, graspers, an electrosurgical knife, a suction tube, an ultrasonic coagulation incision device, a hemostatic device, a radiofrequency cautery device, a medical stapler, a needle driver, an endoscope, a thoracoscope, a laparoscope, and the like. For example, the surgical instrument shaft 119 corresponds to a shaft portion.

The linear motion robot arm 120 includes a first arm 121, a second arm 122, and a third arm 123. A robotic medical instrument 124 is attached to the distal end of the linear motion robot arm 120. The first arm 121 and the second arm 122 are connected by an active joint having a degree of freedom only in horizontal rotation. The second arm 122 and the third arm 123 are connected by an active joint having a degree of freedom only in the horizontal direction. The third arm 123 has a horizontal portion 123A and a vertical portion 123B. The robotic medical instrument 124 is connected to the distal end of the third arm 123 via a gimbal mechanism configured to be rotatable about two axes. The first arm 121 has an active joint having a degree of freedom in the vertical direction (longitudinal direction of the third frame 103). Therefore, the second arm 122 and the third arm 123 are movable in the vertical direction by the first arm 121. Consequently, the robotic medical instrument 124 is allowed to move three-dimensionally, an insertion angle and an insertion depth of the robotic medical instrument 124 into the body cavity are thus controlled.

The robotic medical instrument 124 includes an endoscope holder 125 and an endoscope 126. The endoscope holder 125 is connected to the gimbal mechanism at the distal end of the third arm 123. The endoscope 126 having a shaft shape is connected to the endoscope holder 125. For example, the endoscope 126 corresponds to the shaft portion.

The surgery support device 200 illustrated in FIG. 1 is not a general console-type master slave, but controls the operation of the robot arm portion 100 based on an operation of a surgical instrument (that is, a handheld medical instrument) used by the surgeon during operation.

The handheld medical instrument 201, which is a surgeon operation portion, is a surgical instrument that is actually moved manually by the surgeon to perform a normal treatment, and is inserted into a body cavity through an outer tube 202 inserted into a small-diameter hole formed on an abdominal wall 153 of the patient 150. The handheld medical instrument 201 has, at a distal end portion thereof, a medical instrument (forceps, graspers, an electrosurgical knife, a suction tube, an ultrasonic coagulation incision device, a hemostatic device, a radiofrequency cautery device, a medical stapler, a needle driver, and the like) to be inserted into a body cavity and used, for example. A position and posture measuring device 203 is attached to the handheld medical instrument 201, and the posture of the handheld medical instrument 201 is measured by a sensor described later. The sensor may be a sensor capable of measuring an absolute position and posture in general six degrees of freedom, or may be a sensor capable of measuring only a relative position and posture from a certain time and position.

The position and posture measuring device 203 is composed of a combination of an inertial sensor capable of measuring a posture in three degrees of freedom and a distance sensor capable of measuring an insertion depth into a body cavity. As the inertial sensor, for example, a general sensor such as an acceleration sensor, an inclination sensor, a gyroscope sensor, or a geomagnetic sensor, or a combination thereof can be used. In addition, as the distance sensor, an encoder using a roller rotated by insertion of the handheld medical instrument 201, a distance meter using light or magnetism, or the like can be used.

A mode switching unit 210 includes an operation member for appropriately switching between operation modes of a surgery support system, and is composed of, for example, a hand switch, a foot switch, and the like. The operation modes include a mode of actually performing a surgical operation by operating the handheld medical instrument 201 for treatment, and a mode of using the handheld medical instrument 201 to operate the robotic medical instrument 117 and the end effector 119A (also simply referred to as a robot operation mode). When the surgeon switches between the operation modes via the mode switching unit 210, the controller 211 switches between the operation modes of the system in accordance with a signal from the mode switching unit 210, and records the current operation mode in a RAM (not illustrated). Note that information on a predetermined voice and a predetermined gesture may be acquired via the mode switching unit 210, and the controller 211 may switch to an operation mode corresponding to the input information.

The controller 211 includes a central processing unit such as a CPU or an MPU, a ROM, and a RAM, and executes a program stored in a recording medium such as the ROM or a nonvolatile memory 212 to control the operation of each block of the surgery support device 200. In addition, the controller 211 acquires current position information such as each link or a joint angle (or information on a distance between joint angles or the like obtained on the basis of the current position information, or the like) from each of the horizontal robot arms 110A and 110B. The controller 211 transmits control information for controlling the movement of the robotic medical instrument 117 and the posture of the end effector 119A to the horizontal robot arms 110A and 110B. Further, the controller 211 acquires information on the current position of each arm or the like from the linear motion robot arm 120. The controller 211 transmits control information for controlling the posture of the endoscope 126 to the linear motion robot arm 120. That is, it can also be said that the controller 211 controls the medical robot system 1.

A display unit 213 includes, for example, a display device such as a liquid crystal display or an organic EL display, and displays an image or video inside the body cavity captured by a laparoscope (not illustrated) inserted into the abdominal wall. In addition, the display unit 213 displays a state display inside the system (including, for example, a numerical value indicating the posture of the robot or the handheld medical instrument 201), an operation screen for operating the system, and the like.

The nonvolatile memory 212 includes a recording medium composed of a semiconductor memory, a magnetic disk, or the like, and stores a program executed by the controller 211, a constant for operation, and the like.

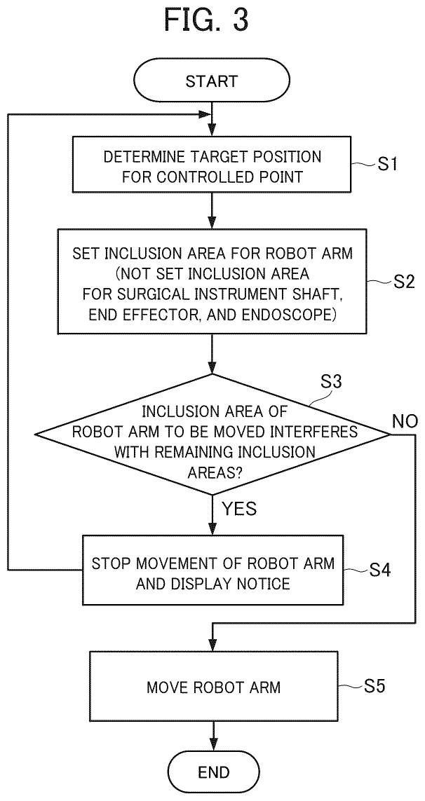

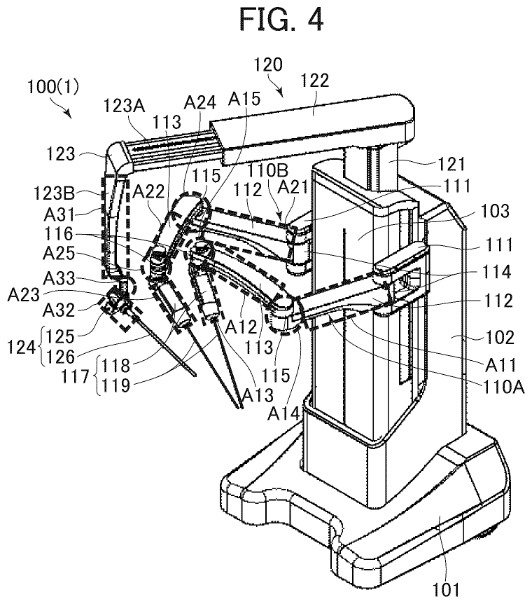

Next, a series of actions related to the operation of the robotic medical instrument 117 or the like using the handheld medical instrument 201 will be described with reference to FIG. 3. FIG. 3 shows a flowchart relating to the operation of the robotic medical instrument 117 and the like using the handheld medical instrument 201 executed by the controller 211. FIG. 4 is an explanatory view illustrating a setting of inclusion areas for the horizontal robot arms 110A and 110B, the linear motion robot arm 120, and the robotic medical instruments 117 and 124 of FIG. 2. The present operation is realized such that the controller 211 loads a program recorded in the nonvolatile memory 212 into a RAM (not illustrated) and executes the program. In addition, the present process is started when, for example, the surgeon operates the mode switching unit 210 to switch the operation mode to the robot operation mode. The robot operation mode is a position control mode for controlling the position of a predetermined portion of, for example, the robotic medical instrument 117 using the handheld medical instrument 201.

In S1, the controller 211 determines a target position to which the end effector 119A of the robotic medical instrument 117 of one horizontal robot arm 110A is moved, on the basis of an amount of each of the insertion angle and the insertion depth changed when the handheld medical instrument 201 is moved by the surgeon. For example, the controller 211 calculates a reference vector directed from a control reference point (the position of the rotation center of the handheld medical instrument 201) to a controlled point at the time when the operation mode is switched to the position control mode. The controlled point refers to a point to be controlled when the surgeon operates the robotic medical instrument 117 and the end effector 119A. The controller 211 stores information on the calculated reference vector in, for example, a RAM or the like. Further, the controller 211 obtains a relative movement amount of the handheld medical instrument 201 from the time when the reference vector is calculated (that is, the time when the operation mode is switched to the position control mode), and determines the target position indicating the position where the controlled point should be located by moving the reference vector on the basis of the obtained relative movement amount.

In S2-S3, the controller 211 determines whether or not one horizontal robot arm 110A collides with a part of any of the other horizontal robot arm 110B, the linear motion robot arm 120, and the robotic medical instruments 117 and 124 when the one horizontal robot arm 110A is controlled to move the end effector 119A to the target position.

In S2, the controller 211 sets an inclusion area (three-dimensional shape (three-dimensional coordinate data)) including an area occupied by each of the horizontal robot arms 110A and 110B, the linear motion robot arm 120, and the robotic medical instruments 117 and 124 at the respective current positions. Specifically, as illustrated in FIG. 4, inclusion areas A11 to A15 and A21 to A25 including respective areas occupied by the second link 112, the third link 113, the second joint 115, the third joint 116, and the actuator unit 118 of each of the horizontal robot arms 110A and 110B are calculated. The inclusion areas A11 to A13 and A21 to A23 each corresponding to the second link 112, the third link 113, or the actuator unit 118 are cylindrical inclusion areas, and the inclusion areas A14, A15, A24, and A25 each corresponding to the second joint 115 or the third joint 116 are spherical inclusion areas. The diameters of the inclusion areas A11 to A15 and A21 to A25 are respectively set to minimum diameters encompassing the second link 112, the third link 113, and the actuator unit 118. The diameters of the inclusion areas A14, A15, A24, and A25 are respectively set to minimum diameters encompassing the second joint 115 and the third joint 116.

Similarly, inclusion areas A31 to A33 including respective areas occupied by the vertical portion 123B of the third arm 123 of the linear motion robot arm 120, the gimbal mechanism at the distal end of the third arm 123, and the endoscope holder 125 are calculated. The inclusion areas A31 and A32 each corresponding to the vertical portion 123B of the third arm 123 or the endoscope holder 125 are cylindrical inclusion areas, and the inclusion area A33 corresponding to the gimbal mechanism at the distal end of the third arm 123 is a spherical inclusion area. The diameters of the inclusion areas A31 and A32 are respectively set to minimum diameters encompassing the vertical portion 123B of the third arm 123, and the endoscope holder 125. The diameter of the inclusion area A33 is set to a minimum diameter encompassing the gimbal mechanism at the distal end of the third arm 123.

In S2, the controller 211 does not set an inclusion area for the surgical instrument shaft 119 and the end effector 119A of each of the horizontal robot arms 110A and 110B and the endoscope 126 of the linear motion robot arm 120. That is, the controller 211 sets the inclusion areas in a portion on the side closer to each of the robot arms 110 and 120 with respect to the shaft portion (the surgical instrument shaft 119 and the endoscope 126) of each of the robot arms 110 and 120 and each of the medical instruments 117 and 124, and does not set an inclusion area in the shaft portion (the surgical instrument shaft 119 and the endoscope 126) of each of the medical instruments 117 and 124 and in portions on the distal end side with respect to the shaft portions (the surgical instrument shaft 119 and the endoscope 126).

In S3, the controller 211 determines whether or not each of the inclusion areas A11 to A15 of the horizontal robot arm 110A set in S2 interferes with the remaining inclusion areas A22 to A25 and A31 to A33 by a known method when one horizontal robot arms 110A is controlled to move the end effector 119A to the target position.

As one example, a method of determining whether or not the inclusion area A11 of the horizontal robot arm 110A interferes with the inclusion area A21 of the horizontal robot arm 110B will be described. First, the controller 211 calculates the position of the inclusion area A11 after a lapse of a predetermined time from the start of movement, and determines whether or not the inclusion area A11 at the position interferes with the inclusion area A21. For example, the controller 211 determines whether the line segment of the center axis of the inclusion area A11 having a cylindrical shape and the line segment of the center axis of the inclusion area A21 having a cylindrical shape match any of the following patterns.
Pattern 1: there is a straight line orthogonal to both of the line segments, and the distance between the intersections thereof is the shortest distance.
Pattern 2: in a case other than the pattern 1, the length of a vertical line drawn from an end point of one of the line segments to the other line segment is the shortest distance.
Pattern 3: in a case other than the patterns 1 and 2, the distance between end points of the line segments is the shortest distance.

If the case of the pattern 1, when the shortest distance l between the line segments is smaller than the sum of the radii r1 and r2 of the inclusion areas A11 and A21 (l≤r1+r2), it is determined that the inclusion area A11 interferes with the inclusion area A21.

In the case of the pattern 2 or the pattern 3, when the shortest distance l is smaller than the sum of the radii r1 and r2 of the inclusion areas A11 and A21 (l≤r1+r2), the controller 211 determines whether or not interference occurs through the following procedures 1 to 3.
Procedure 1: straight lines respectively obtained by extending the line segments are assumed, and a straight line orthogonal to both of the straight lines is obtained.
Procedure 2: since the case where the intersections between the orthogonal straight line and the respective line segments are not on any of the line segments of the center axes corresponds to the pattern 3, it is determined that interference occurs when the distance between end points is smaller than the sum of the radii r1 and r2.
Procedure 3: since the case where the intersections between the orthogonal straight line and the respective line segments are on the line segment of the center axis of either one of the inclusion areas corresponds to the pattern 2, it is determined that interference occurs when a vertical line is drawn from an end point of the line segment on which the intersections are not present to the line segment of the other center axis, and the length thereof is smaller than the sum of the radii r1 and r2.

In this manner, the controller 211 repeatedly determines whether or not the inclusion area A11 interferes with the inclusion area A21 at predetermined time intervals. When the controller 211 determines that at least one of the inclusion areas A11 to A15 interferes with any of the remaining inclusion areas A22 to A25 and A31 to A33 (S3: YES), the process proceeds to S4. On the other hand, when the controller 211 determines that the inclusion areas A11 to A15 do not interfere with the remaining inclusion areas A22 to A25 and A31 to A33 (S3: NO), the process proceeds to S5.

In S4, the controller 211 stops the movement of the horizontal robot arm 110A, and displays on the display unit 213 a notice indicating that the horizontal robot arm 110A interferes with the horizontal robot arm 110B or the linear motion robot arm 120. Further, the controller 211 displays a message prompting the surgeon to perform an operation to move the end effector 119A with the handheld medical instrument 201 again, and the process returns to S1.

In S5, the controller 211 moves the end effector 119A of the horizontal robot arm 110A to the target position, and ends the position control mode.

According to the medical robot system 1 of the present embodiment, the controller 211 sets an inclusion area in a portion on the side closer to each of the robot arms 110 and 120 with respect to the shaft portion (the surgical instrument shaft 119 and the endoscope 126) of each of the robot arms 110 and 120 and each of the robotic medical instruments 117 and 124, and does not set an inclusion area in the shaft portion (the surgical instrument shaft 119 and the endoscope 126) of each of the medical instruments 117 and 124 and in portions on the distal end side with respect to the shaft portions (the surgical instrument shaft 119 and the endoscope 126). Then, the controller 211 determines whether or not the inclusion areas A11 to A15 after moving the inclusion areas A11 to A15 corresponding to one robot arm 110A and the robotic medical instrument 117 attached thereto interfere with the inclusion areas A22 to A25 and A31 to A33 corresponding to the remaining robot arms 110B and 120 and the medical instruments 117 and 124 attached thereto. Consequently, interference of the robot arms 110 and 120 can be prevented while ensuring freedom of movement of the robotic medical instruments 117 and 124 located in the abdominal wall 153 (in the body cavity).

Since the handheld medical instrument 201 has a medical instrument that is directly used by a surgeon and inserted into a body cavity, by controlling each of the robotic medical instruments 117 and 124 with the medical instrument operated by the surgeon, each of the robotic medical instruments 117 and 124 can be precisely moved, and accuracy of an operative procedure can be improved.

Since the inclusion areas A11 to A13 and A21 to A23 respectively corresponding to the links 111 to 113 have a cylindrical shape, and the inclusion areas A14, A15, A24, and A25 respectively corresponding to the joints 114 and 115 have a spherical shape, the calculation speed of the controller 211 can be improved.

The technology disclosed in the present specification is not limited to the above-described embodiments, and can be modified to have various forms without departing from the spirit thereof, and for example, the following modification is also possible.

For example, the number of robot arms is not limited to that in the above embodiments, and may be two or four or more.

What is claimed is:

1. A medical robot system, comprising:
a plurality of robot arms, each having a medical instrument attached thereto;
a surgeon operation portion that is configured to be directly operated by a surgeon so as to operate the robot arms and the medical instruments; and
a controller that is configured to control the robot arms and the medical instruments based on an operation of the surgeon operation portion operated by the surgeon, wherein
each of the medical instruments have a shaft portion inserted into a body cavity, and
when an instruction to operate one of the robot arms and the medical instrument attached thereto is received,
for each of the robot arms and each of the medical instruments attached thereto, the controller is configured to set an inclusion area encompassing the robot arm and a portion on a side closer to the robot arm with respect to the shaft portion, and does not set an inclusion area encompassing the shaft portion and a portion on a distal end side with respect to the shaft portion, and
the controller is configured to determine whether a first inclusion area, after moving the first inclusion area corresponding to a first robot arm and the medical instrument attached thereto, interferes with a second inclusion area corresponding to a second robot arm and the medical instruments attached thereto.

2. The medical robot system according to claim 1, wherein the surgeon operation portion has a medical instrument directly used by the surgeon and inserted into a body cavity.

3. The medical robot system according to claim 1, wherein
the robot arm has a plurality of links and a joint connecting adjacent links, and
the inclusion area corresponding to each of the links has a cylindrical shape, and the inclusion area corresponding to the joint has a spherical shape.

4. The medical robot system according to claim 1, wherein
the first robot arm has a first link, a second link, and a first joint connecting the first link and the second link,
when the instruction to operate the first robot arm and the medical instrument attached thereto is received, the controller is configured to individually set, as a part of the inclusion area corresponding to the first robot arm and the medical instrument attached thereto, a first link inclusion area encompassing the first link, a second link inclusion area encompassing the second link, and a first joint inclusion area encompassing the first joint, and
the controller is configured to determine whether or not each of the first link inclusion area, the second link inclusion area, and the first joint inclusion area after moving the inclusion area corresponding to the first robot arm and the medical instrument attached thereto interferes with the inclusion area corresponding to the second robot arm and the medical instrument attached thereto.

5. A method of controlling a medical robot system that includes a plurality of robot arms, each having a medical instrument attached thereto, a surgeon operation portion that is configured to be directly operated by a surgeon so as to operate the robot arms and the medical instruments, and a controller that is configured to control the robot arms and the medical instruments based on an operation of the surgeon operation portion operated by the surgeon, each of the medical instruments having a shaft portion inserted into a body cavity, the method comprising:
upon receiving an instruction to operate one of the robot arms and the medical instrument attached thereto,
setting, for each of the robot arms and each of the medical instrument attached thereto, an inclusion area encompassing the robot arm and a portion on a side closer to the robot arm with respect to the shaft portion, and not setting an inclusion area encompassing the shaft portion and a portion on a distal end side with respect to the shaft portion; and
determining whether a first inclusion area, after moving the first inclusion area corresponding to a first robot arm and the medical instrument attached thereto, interferes with a second inclusion area corresponding to a second robot arm and the medical instruments attached thereto.

6. A non-transitory computer-readable medium storing instructions that, when executed by a controller of a medical robot system that includes a plurality of robot arms, each having a medical instrument attached thereto, a surgeon operation portion that is configured to be directly operated by a surgeon so as to operate the robot arms and the medical instruments, and the controller that is configured to control the robot arms and the medical instruments based on an operation of the surgeon operation portion operated by the surgeon, each of the medical instruments having a shaft portion inserted into a body cavity, cause the controller to execute operations comprising:
receiving an instruction to operate one of the robot arms and the medical instrument attached thereto;
setting, for each of the robot arms and each of the medical instruments attached thereto, an inclusion area encompassing the robot arm and a portion on a side closer to the robot arm with respect to a shaft portion, and refraining from setting an inclusion area encompassing the shaft portion and a portion on a distal end side with respect to the shaft portion; and
determining whether a first inclusion area, after being moved corresponding to a first robot arm and the medical instrument attached thereto, interferes with a second inclusion area corresponding to a second robot arm and the medical instruments attached thereto.
